(19) United States
(12) Patent Application Publication
(10) Pub. No.: US 2023/0190687 A1
(43) Pub. Date: Jun. 22, 2023

(54) SODIUM BUTYRATE FOR USE IN THE PREVENTION OR TREATMENT OF RHINOVIRUS INFECTION

(71) Applicant: EDINBURGH NAPIER UNIVERSITY, Edinburgh (GB)

(72) Inventors: Craig Stevens, Edinburgh (GB); Peter Barlow, Edinburgh (GB)

(21) Appl. No.: 17/915,925

(22) PCT Filed: Mar. 31, 2021
(86) PCT No.: PCT/GB2021/050809

§ 371 Date: Sep. 29, 2022

(30) Foreign Application Priority Data
Mar. 31, 2020 (GB) 2004690.0

(51) Int. Cl. A61K 31/19; A61K 31/59; A61K 9/00; A61K 9/127; A61K 9/50

(52) U.S. Cl. CPC A61K 31/19; A61K 31/59; A61K 9/0073; A61K 9/127; A61K 9/5068

(57) ABSTRACT

A composition comprising sodium butyrate for the prevention and/or treatment of a viral infection caused by Rhinovirus. The composition may further comprise Vitamin D. The composition may be administered in the form of a tablet or capsule, in the form of an inhaler, or provided as a dietary supplement.

The invention relates to a composition for use in the prevention and/or treatment of an infection caused by Rhinovirus.

Rhinovirus is a primary cause of the common cold and is considered to be one of the most common viral infectious agents in humans. There are around 160 different serotypes of Rhinovirus that can infect humans that differ according to their protein composition. Acute Rhinovirus infection is a viral upper respiratory tract infection.

At present, there are no effective vaccines for the prevention of Rhinovirus infection in humans. Typically, children get infected with Rhinovirus around 8 to 12 times per year and adults get infected around 3 to 4 times per year. The approximate cost of Rhinovirus to the US economy is estimated to be around $40 billion every year.

Individuals suffering from the common cold or similar viral infections often take supportive treatments like pain killers or decongestants to relieve their symptoms. In addition, patients often purchase over the counter supplements such as those comprising Vitamin C, zinc and other substances that are thought to have an effect on enhancing the immune response. Examples of such supplements are the products ‘Airborne’ or ‘Zicam’. Immune support products are part of a $500 million industry that is growing at around 5 to 6 percent per year.

The present invention seeks to address the above problems.

According to a first aspect, there is provided a composition comprising sodium butyrate for use in the prevention, amelioration and/or treatment of a viral infection caused by Rhinovirus.

Preferably, there is provided a composition comprising sodium butyrate for the prevention and/or treatment of acute Rhinovirus infection. Preferably, there is provided a composition comprising sodium butyrate for the prevention and/or treatment of a viral upper respiratory tract infection. Preferably, the composition comprises sodium butyrate having the formula C4H7NaO2 (or C₄H₇NaO₂).

Advantageously, the use of the composition comprising sodium butyrate results in the reduction and/or removal of Rhinovirus infection within an individual.

Advantageously, the use of the composition may have a prophylactic effect in relation to a viral infection caused by Rhinovirus.

Typically, the composition of the present invention acts through the stimulation of the immune response within an individual. Preferably, the composition stimulates the immune cells of an individual to release one or more substances that kill Rhinovirus and/or inhibit its replication.

Advantageously, the use of the composition comprising sodium butyrate does not induce antimicrobial resistance within an individual since it works through stimulation of the innate immune response, rather than directly targeting the virus itself.

Advantageously, the composition of the present invention is easy to produce and cheap to purchase. Advantageously, the composition of the present invention does not produce any side effects within a patient when taken within recommended dosages.

In another embodiment, the composition further comprises Vitamin D. In one embodiment, the use of a composition comprising sodium butyrate and Vitamin D reduces and/or removes the Rhinovirus infection within an individual. Typically, the combination of sodium butyrate and Vitamin D stimulates the immune response of a patient and reduces and/or removes the Rhinovirus infection from the patient.

Typically, the composition comprises between 100 mg and 300 mg sodium butyrate. In one embodiment, the composition may comprise approximately 2000 mg sodium butyrate.

Typically, the composition further comprises between 400 IU and 4000 IU (i.e. between 10 μg and 100 μg) Vitamin D.

It is preferred that the composition is administered as a single dose per day.

In one embodiment, the composition is provided as a dietary supplement. Advantageously, the provision of the composition as a dietary supplement allows an individual to boost their own innate immune response to the virus. Advantageously, dietary supplementation with the composition comprising sodium butyrate and/or Vitamin D enhances the host immune response to Rhinovirus.

Advantageously, sodium butyrate is a naturally occurring substance (a short chain fatty acid) that may be available to purchase from pharmacies and/or chemists, or may be prescribed to patient as a supplement. Advantageously, sodium butyrate may be taken over the counter with no documented side effects within recommended doses.

In one embodiment, the composition may be administered by mouth. In a preferred embodiment, the composition may be administered in the form of a tablet or capsule. In another embodiment, the composition may be administered in the form of an inhaler, such as a nasal inhaler. Preferably, the composition is administered as a single dose, once per day.

In one embodiment, the composition may be coated with a material such as a lipid or sugar, or a combination thereof. Typically, the coating or target-guiding material may dissolve to allow efficient and sufficient delivery of the composition to cells, tissues or organs.

In one embodiment, the composition may be incorporated into a food or drink.

In one embodiment, the composition may comprise at least one acceptable carrier and may be carried within a vesicle, micelle, liposome, nanoparticle or other suitable vehicle.

According to a second aspect, there is provided a dietary supplement comprising a composition in accordance with the first aspect of the invention.

According to a third aspect, there is provided a tablet comprising a composition in accordance with the first aspect of the invention.

According to a fourth aspect, there is provided an inhaler comprising a composition in accordance with the first aspect of the invention.

In one embodiment, the inhaler is a nasal inhaler.

According to a fifth aspect, there is provided a method for preventing, ameliorating and/or treating a viral infection caused by Rhinovirus using a composition comprising sodium butyrate in accordance with the first aspect of the invention.

Preferably, there is provided a method for preventing, ameliorating and/or treating acute Rhinovirus infection using a composition comprising sodium butyrate in accordance with the first aspect of the invention.

In one embodiment the method comprises the use of a composition comprising sodium butyrate and Vitamin D.

The invention will be further described by way of example and with reference to the following FIGURES, in which:

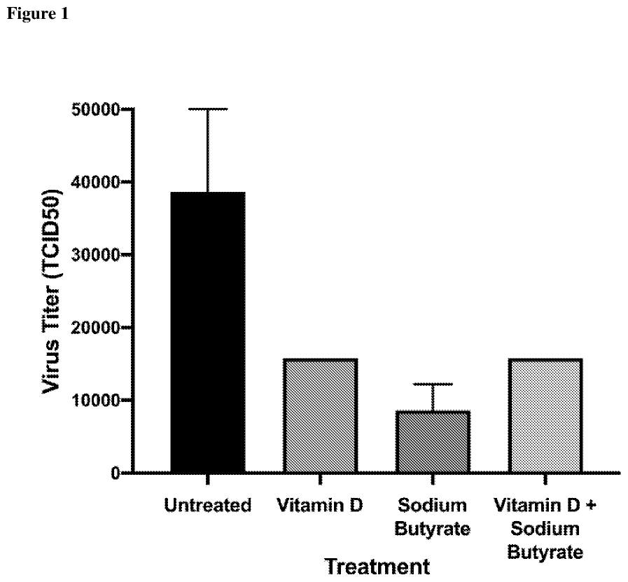

FIG. 1 is a graph showing the effect on cells that have been infected with Rhinovirus following treatment with sodium butyrate, Vitamin D or a combination of sodium butyrate and Vitamin D.

Advantageously, the use of the composition comprising sodium butyrate and/or Vitamin D results in the reduction and/or removal of Rhinovirus infection within an individual.

The composition in accordance with the present invention acts through the stimulation of the immune response within an individual, wherein the composition stimulates the immune cells of an individual to release one or more substances that kill/inactivate Rhinovirus and/or inhibit its replication.

In one embodiment, the composition is provided as a dietary supplement. Advantageously, dietary supplementation with sodium butyrate enhances the host's immune response to Rhinovirus. Preferably, the composition is provided in the form of a dietary supplement in pill form and marketed as an over the counter dietary supplement.

In another embodiment, the composition may be administered in the form of a tablet or capsule. In this embodiment, the composition may be coated with a material such as a lipid or sugar, or a combination thereof. Typically, the coating or target-guiding material dissolves to allow efficient and sufficient delivery of the conjugate to the cells, tissues or organs.

Pharmaceuticals, food supplements or dietary supplements often contain compounds and/or other ingredients derived from a natural source or from a synthetic source. The natural or synthetic ingredient may be an active ingredient that provides a beneficial effect or may be an inactive ingredient.

Inactive ingredients, often termed excipients, are typically present within a pharmaceutical or a food or dietary supplement formulation. The choice of excipient or excipients in a formulation is dependent upon whether the active ingredients are to be delivered in a tablet form, a capsule form, in a liquid form or the manufacturing process.

The active or inactive ingredient may be a compound considered as “Generally Regarded As Safe” (GRAS) for human or animal consumption.

Excipients may be derived from a natural source or from a synthetic source. Excipients have a wide range of physical or chemical properties that are dependent on their structure. Excipient properties include but are not limited to anti-adhesion, binding, bulking, providing a protective or enteric coating, aiding dissolution or disintegration, providing a colouring, a flavouring or a sweetening effect, providing a flow or glidant effect, solvation, a sorbent effect or a lubricating effect that is beneficial in formulation, manufacturability or the delivery of the active ingredients.

An example of the composition according to the invention is described below and contains both active ingredients and excipient ingredients that are common within pharmaceuticals and food or dietary supplements:

Active Ingredients:

| | | |
|---|---|---|
| Vitamin D (CAS No: 69-97-0) | 25 | micrograms (1000 IU or 25 ug) |
| Sodium Butyrate | 300 | milligrams (mg) |
| (CAS No: 156-54-7) | | |

Excipients/Inactive Ingredients:

| | | |
|---|---|---|
| Microcrystalline Cellulose | 350 | milligrams (mg) |
| (CAS No: 9004-34-6) | | |
| Calcium Diphosphate (CAS no: 7758-87-4) | 300 | milligrams (mg) |
| Silica SiO2 (CAS No: 7631-86-9) | 20 | milligrams (mg) |
| Magnesium Stearate (CAS No: 557-04-0) | 30 | milligrams (mg) |

The above example formulation is for a single, solid dose to be given orally.

Those skilled in the art will understand the benefit in varying the ratio of the ingredients within the above example formulation to enable volume manufacturing of tablets or capsules for human or animal consumption.

In another embodiment, the composition may be prepared comprising sodium butyrate as an active ingredient and the excipients/inactive ingredients microcrystalline cellulose, calcium diphosphate, silica SiO₂ and magnesium stearate in the amounts as mentioned above.

With reference to FIG. 1, an experiment was carried out to treat leukocytes (the human monocytic THP-1 macrophage cell line differentiated into macrophages) with sodium butyrate, Vitamin D, or with a combination of sodium butyrate and Vitamin D.

The leukocyte cells were incubated for a period of time to allow them to release proteins into the surrounding cell culture media (referred to as ‘conditioned media’). The conditioned media was then incubated with HRV1B Rhinovirus. The leukocyte cells were incubated with a solution comprising 0.5 mM sodium butyrate, a solution comprising 0.5 mM Vitamin D, and with a solution comprising both 0.5 mM sodium butyrate and 0.5 mM Vitamin D.

The results shown in FIG. 1 demonstrate that incubation with sodium butyrate resulted in a 75-80% reduction in measurable live virus, when compared with the control (treated with distilled water). In addition, incubation with Vitamin D alone, or with sodium butyrate and Vitamin D together resulted in a significant reduction in measureable live virus when compared with the control.

The data shown is an average of n=3 independent biological replicates (the titration is an average of ten fold reductions quantified over the three replicates).

Sodium butyrate and Vitamin D have effects on different intracellular mediators and/or signalling molecules in order to provide an effective response to viral infection. Thus, the provision of a composition comprising both sodium butyrate and Vitamin D would be advantageous since some individuals may be more responsive, in the context of their innate immunity, to either sodium butyrate or Vitamin D. Thus, administering a composition comprising both sodium butyrate and Vitamin D would provide an effective reduction in viral infection in a broader range of people.

Butyrate has been shown to increase cathelicidin production (Schauber et al, Gut 2003, 52(5), 735-741 and Hase et al, Infect Immun 2002, 70 (2), 953-963).

With reference to the present invention, it is understood that the sodium butyrate reduces the level of Rhinovirus titre through the stimulation of cathelicidins.

In addition, downregulation of inflammation in the context of viral infection can be an additional beneficial outcome since in Rhinovirus infection, much of the symptomology can be attributed to virus induced inflammation (Greenberg, Arch Int Med, 2003 163(3), 278-284). It has been shown in mouse models of influenza infection that exogenous LL-37 (a human member of the cathelicidin family) can decrease inflammation (Barlow et al, PLos One, 2011, 6(10)), thus resulting in enhanced survival rates. It has also been demonstrated that exogenous LL-37 can reduce Rhinovirus induced inflammatory cytokine release in cell models (Casanova et al, Front Immunol 2020, 11 (85).

With reference to the present invention, it is understood that sodium butyrate can reduce inflammation caused by viral infection through a reduction in NFkappa B signalling which would be of benefit for relief of the symptoms of Rhinovirus infection.

1. A composition comprising sodium butyrate for use in the prevention and/or treatment of a viral infection caused by Rhinovirus.

2. A composition according to claim 1, for use in the prevention and/or treatment of an acute Rhinovirus infection.

3. A composition according to claim 1 or claim 2, wherein the composition stimulates the immune cells of an individual to release substances that kill Rhinovirus and/or inhibit its replication.

4. A composition according to any preceding claim, wherein the composition further comprises Vitamin D.

5. A composition according to any preceding claim, wherein the composition is administered by mouth.

6. A composition according to claim 8, wherein the composition is administered in the form of a tablet or capsule.

7. A composition according to any one of claims 1 to 4, wherein the composition is administered in the form of an inhaler.

8. A composition according to any one of claims 1 to 6, wherein the composition is coated with a material such as a lipid or sugar, or a combination thereof.

9. A composition according to any one of the preceding claims, wherein the composition comprises at least one acceptable carrier and may be carried within a vesicle, micelle, liposome, nanoparticle or other suitable vehicle.

10. A composition according to any preceding claim, wherein the composition is provided as a dietary supplement.

11. A composition according to any preceding claim, wherein the composition is incorporated into a food or drink.

12. A dietary supplement comprising a composition comprising sodium butyrate for the prevention and/or treatment of a viral infection caused by Rhinovirus.

13. A dietary supplement according to claim 12, wherein the composition further comprises Vitamin D.

14. A tablet comprising a composition comprising sodium butyrate for the prevention and/or treatment of a viral infection caused by Rhinovirus.

15. A tablet according to claim 14, wherein the composition further comprises Vitamin D.

16. An inhaler comprising a composition comprising sodium butyrate for the prevention and/or treatment of a viral infection caused by Rhinovirus.

17. An inhaler according to claim 16, wherein the composition further comprises Vitamin D.

18. An inhaler according to claim 16 or 17, wherein the inhaler is a nasal inhaler.

19. A method for preventing and/or treating a viral infection caused by Rhinovirus using a composition comprising sodium butyrate in accordance with any one of claims 1 to 11.

20. A method according to claim 19, for the prevention and/or treatment of acute Rhinovirus infection using a composition comprising sodium butyrate in accordance with any one of claims 1 to 11.

21. A method according to claim 19 or 20, comprising the use of a composition comprising sodium butyrate and Vitamin D.
